(19) United States
(12) Patent Application Publication
(10) Pub. No.: US 2003/0053980 A1
(43) Pub. Date: Mar. 20, 2003

(54) Shaving compositions containing highly lubricious water soluble polymers

(75) Inventors: Kenneth T. Dodd, Upton, MA (US); John V. Lawler, N. Potomac, MD (US); Christopher S. Coughlin, Leonardtown, MD (US)

Correspondence Address: PATENT AND TRADEMARK COUNSEL, THE GILLETTE COMPANY, 800 BOYLSTON STREET, BOSTON, MA, 02199, US

(73) Assignee: The Gillette Company

(21) Appl. No.: 10/101,004

(22) Filed: Mar. 18, 2002

Related U.S. Application Data
(60) Provisional application No. 60/287,619, filed on Apr. 30, 2001.

(51) Int. Cl. A61K 7/15

(52) U.S. Cl. 424/73

(57) ABSTRACT

The present invention is directed to shaving compositions which comprise one or more water soluble polymers to increase lubricity and enhance shaving performance. The polymers employed in the shaving compositions provide the composition with a stress ratio (i.e., the ratio of normal stress to shear stress) in excess of 3.0 when measured at a shear rate of 800 sec⁻¹. A preferred combination of polymers includes polyethylene oxide (mol. Wt.≧1 million) and natural or synthetic polysaccharide gum. The present invention is also directed to an improved shaving method in which such compositions are applied to an area of skin, then said area is shaved. In another aspect, the present invention embraces shaving compositions comprising water, a cleansing or conditioning agent for hair or skin, polyethylene oxide (mol. Wt.≧1 million) and natural or synthetic polysacchride gum.

### BACKGROUND OF THE INVENTION

This invention relates to improved shaving compositions which contain a water soluble polymer or a combination of water soluble polymers, such as, for example, polyethylene oxide and natural or synthetic gum, to increase lubricity and enhance shaving performance. It also relates to improved shaving methods using such compositions.

Polyethylene oxide is a lubricious polymer which is well-known for use in shaving. It has been included in a variety of compositions which can be applied either before or during shaving. For example, solid shaving compositions containing polyethylene oxide are disclosed in U.S. Pat. No. 3,956,951 (Jennings), U.S. Pat. No. 4,381,293 (Michel) and U.S. Pat. No. 5,431,906 (Mohseni). These solid compositions are rubbed on or applied to the face prior to shaving. Razor cartridges with lubricating strips that contain polyethylene oxide are disclosed, for example, in U.S. Pat. No. 4,170,821 (Booth), U.S. Pat. No. 5,113,585 (Rogers), U.S. Pat. No. 5,345,680 (Vreeland) and WO 96/13360 (Tseng). These lubricating strips leach out polyethylene oxide upon contact with water during shaving to provide lubrication and improve shaving comfort.

Aqueous solutions containing polyethylene oxide and a humectant polyol such as glycerol are disclosed in U.S. Pat. No. 5,342,617 (Gold) for use as a vaginal lubricant or a shaving lubricant. Shave creams, foams and gels containing polyethylene oxide are disclosed, for example, in U.S. Pat. No. 4,999,183 (Mackles), U.S. Pat. No. 5,248,495 (Patterson), U.S. Pat. No. 5,326,556 (Barnet), U.S. Pat. No. 5,500,211 (George), U.S. Pat. No. 5,560,859 (Hartmann) and U.S. Pat. No. 5,858,343 (Szymczak). Many of the compositions disclosed in these patents also include a gelling aid or thickening agent such as hydroxyalkyl cellulose. While some of these patents suggest generally that gums may optionally be included in the compositions (presumably for their known thickening properties), none of them specifically disclose a composition containing a gum, nor do they suggest that both a gum and polyethylene oxide should be used together. In fact, Szymczak suggests that his shave gel compositions may be prepared free of gelling aids.

Cosmetic compositions containing natural or synthetic gums as thickening agents or gelling aids are also well known. For example, U.S. Pat. No. 4,145,411 (Mende) suggests the possible use of gum thickening agents (e.g. carrageenan) in shaving foams. FR 2,555,443 (Spinoglio) discloses an acidic shave cream preparation containing xanthan gum. U.S. Pat. No. 4,871,530 (Grollier) discloses a delayed-foaming gel which contains a heterobiopolysaccharide such as xanthan gum to prevent the foaming agent from separating out, particularly in the presence of cationic hair treatment compounds. U.S. Pat. No. 5,635,171 (Nadaud) discloses cosmetic gel compositions thickened with a combination of a carboxyvinyl polymer, such as Carbopol, and a polygallactomannan, such as carob gum, guar gum or tara gum. However, xanthan gum is specifically excluded from such compositions as not providing the desired result.

In U.S. Pat. No. 4,242,098 (Braun), aqueous coal slurries are disclosed which contain a higher weight percent of solids as a result of the inclusion of small amounts of certain water-soluble polymers. The list of useful polymers includes polyethylene oxide, hydroxyethyl cellulose, xanthan gum and hydroxypropyl guar gum. GB 2,047,736 (Kuan) discloses a tire lubricant composition which contains water, ethylene glycol, polyethylene oxide and xanthan gum. U.S. Pat. No. 3,919,092 (Norton) discloses aqueous compositions of polyethylene oxides and polysaccharide gums, such as xanthan gum, as displacing fluids used in oil recovery. U.S. Pat. No. 5,024,676 (Moriyama) discloses super-heavy oil emulsion fuel which contains, among other things, a hydrophilic polymer, which may be a gum such as xanthan gum, and a synthetic polymer, which may be a polyethylene oxide. U.S. Pat. No. 5,228,909 (Burdick) discloses fluidized polymer suspensions which contain xanthan gum and a polymer such as hydroxyethyl cellulose or polyethylene oxide. U.S. Pat. No. 5,543,443 (Rajaiah) discloses denture adhesive compositions which may contain sodium carboxymethyl cellulose gum and a polyethylene oxide.

### SUMMARY OF THE INVENTION

The present invention is directed to shaving compositions which comprise one or more water soluble polymers to increase lubricity and enhance shaving performance. The polymers employed in the shaving compositions provide the composition with a stress ratio (i.e., the ratio of normal stress to shear stress) in excess of 3.0, preferably in excess of 4.0, most preferably in excess of 5.0 when measured at a shear rate of 800 sec⁻¹. It has been found that shaving compositions with significant stress ratios provide enhanced shaving performance. A preferred combination of polymers includes polyethylene oxide (mol. wt.≧1 million) and natural or synthetic gum. The present invention is also directed to an improved shaving method in which such compositions are applied to an area of skin, then said area is shaved. It has been discovered that certain high molecular weight water soluble polymers, and particularly certain combinations of polymers, which impart the aforementioned stress ratio, are highly lubricious and, therefor, ideal for inclusion in shaving compositions, including aqueous compositions such as shave lotions, creams, foams and gels.

In another aspect, the present invention embraces shaving compositions comprising water, polyethylene oxide (mol. wt.≧1 million) and natural or synthetic gum. Preferably these shaving compositions will additionally comprise a water dispersible (or soluble) surface active agent. More preferably, these shaving compositions will comprise a propellant or a self-foaming agent so that they can be dispensed as a foam or as a self-foaming lotion or gel.

### DETAILED DESCRIPTION OF THE INVENTION

The shaving compositions of the present invention are aqueous compositions and may be in the form of liquids, lotions or gels. These compositions will include a water soluble polymer or a combination of water soluble polymers which provide the final composition with a stress ratio (i.e., the ratio of normal stress to shear stress measured at a shear rate of 800 sec⁻¹) in excess of 3.0, preferably in excess of 4.0, most preferably in excess of 5.0. These shaving compositions are substantially more lubricious than commercially available shaving compositions and provide a substantially more comfortable shave.

The term stress ratio, when used throughout this specification, is intended to mean the ratio of normal stress to shear stress. For purposes of the present invention, these values should be measured at (or interpolated to) a shear rate of 800 sec⁻¹. The measurements of normal stress and shear stress are standard rheological procedures made with a rotational rheometer such as an AR-1000 (TA Instruments), an ARES (Rheometrics Scientific), or an SR-2000 (Rheometrics Scientific). Measurement of normal stress and shear stress preferably should be made at a shear rate of 800 sec⁻¹. However, if the nature of the solution to be measured makes it difficult or impossible to measure at that shear rate, then the measurement may be made at another shear rate and the values interpolated or extrapolated to 800 sec⁻¹ in accordance with known methods. The stress ratio of a shaving composition is ideally measured before the addition of propellant or self-foaming agent. However, the stress ratio of a finished shaving composition (i.e., one containing propellant or self-foaming agent) may be measured after first allowing the foam to collapse and the propellant or self-foaming agent to evaporate. This can be readily accomplished by blending the composition with a small amount of water (e.g., three parts composition to one part water), then placing this mixture in a sealed container at about 45° C. for 24-48 hours.

Suitable water soluble polymers or combinations of water soluble polymers that may be advantageously utilized in the shaving compositions of the present invention may be readily found by incorporating candidate polymers or polymer combinations into a preselected shaving composition, then measuring the stress ratio of that shaving composition to determine if it exceeds 3.0, preferably 4.0. Any water soluble polymer or polymer combination which imparts the aforementioned stress ratio to the shaving composition may be utilized. If desired, potential candidate polymers or polymer combinations may be initially screened by measuring the stress ratio of simple aqueous solutions of such polymers or polymer combinations. However, the stress ratio of any final shaving preparation may vary from such an initial screening depending on the nature of other components in the composition. Therefor, ultimately the final shaving composition must be tested to insure that it satisfies the aforementioned criteria. When screening candidate polymers in simple aqueous solutions, one will typically select a concentration, depending on molecular weight and solubility, between 0.01% and 10%, more typically between 0.1% and 5%. It may also be desirable to test an aqueous polymer solution at a dimensionless concentration (which is the product of mass concentration (g/cm³) and intrinsic viscosity (cm³/g)) of 0.1 to 5, preferably 0.5 to 2.5.

Candidate water soluble polymers may be selected from, but are not limited to, those falling within the following polymer types:

High molecular weight water soluble polyethers, such as those of the formula R—(OCH₂—CH₂)ₙ—R′, where R and R′ may be any moiety which does not cause the polymer to become insoluble in water and n is typically 20,000 or more. For example, R and R′ independently may be selected from H, OH, CH₃, CONH2, and COOH. Polyethylene oxide of molecular weight one million or more is preferred.

High molecular weight water soluble vinyl polymers, such as those of the formula —(CH₂—CRR′)ₙ_{⁻}, where R and R′ may be any moiety which can confer water solubility and n is an integer sufficient to provide a molecular weight of one million or higher. For example, R may be H or CH₃ and R′ may be OH, CONH2, pyrrolidone, or COOH. These polymers include polyacrylamides, polyvinylpyrrolidones, polyacrylic acids, polymethacrylic acids and copolymers and terpolymers thereof.

High molecular weight water soluble vinyl copolymers and terpolymers, such as those of the formula —(CH₂—CR₁R₂)ₙ—(CH₂—CR₃R₄)ₘ—(CH₂—CR₅R₆)ₚ_{⁻}, where R₁ to R₆ independently may be selected from any moiety such that the combination of R₁ to R₆ confers water solubility, and where n, m and p are zero or an integer such that n+m+p is sufficient to provide a molecular weight of one million or higher. In other words, at least one of R₁ to R₆ must be water soluble and the total number of such water soluble groups in the molecule must be such as to render the polymer water soluble. Examples include poly(acrylamide-co-acrylic acid), poly(acrylamide-co-2-acrylamido-2-methyl-1-propanesulfonic acid), poly(acrylamide-co-3-acrylamido-3-methylbutanoic acid), poly(methacrylic acid-co-acrylamide), and poly(N-vinylpyrrolidone-co-2-dimethylaminoethyl methacrylate).

A particularly advantageous combination of polymers which may be utilized in shaving compositions includes polyethylene oxide and a water soluble natural or synthetic gum. It has been found that polyethylene oxide and natural or synthetic gum, as described above, interact synergistically to substantially increase the stress ratio of a shaving composition, thereby reducing the coefficient of friction between the razor cartridge and the skin.

The polyethylene oxides which may be used effectively in compositions of the present invention include one or more polyethylene oxides of molecular weight of about one million or higher, typically up to about 5 million. Such polyethylene oxides include, for example, one or more of the following: PEG-23M (WSR N-12K (Union Carbide; M.Wt.≅1,000,000), PEG-45M (WSR N-60K (Union Carbide; M.Wt.≅2,000,000), PEG-90M (WSR-301 (Union Carbide; M.Wt.≅4,000,000), PEG-115M (Polyox Coagulant (Union Carbide; M.Wt.≅5,000,000). If desired, some lower molecular weight polyethylene oxides may be blended with the aforementioned polyethylene oxides of molecular weight one million or higher, although this is not preferred.

The natural or synthetic gums which may be used effectively in compositions of the present invention include one or more polysaccharide gums. These gums usually consist of a hetero- or homo- polysaccharide backbone with varying degrees and types of substitution including, but not limited to, methylation, ethoxylation, propoxylation, sulfonation, or addition of further sugar residues singly or in groups. Substitution may be naturally occurring or synthetically performed or a combination of these. Suitable gums include, but are not limited to, xanthan gum, carrageenan gum, guar gum, locust bean gum, and hydroxypropyl guar gum.

The amount of polymers included in the shaving compositions of the present invention will vary depending on the form of the final composition, but the amount should be such as to provide an effective lubricious layer on the skin, that is a lubriciously effective amount. When the shaving composition is an aqueous composition, such as a shave cream, lotion, foam or gel, it will have a sufficient amount of polymers to provide a stress ratio (composition without propellant) in excess of 3.0, preferably in excess of 4.0, more preferably in excess of 5.0. Typically, aqueous compositions will comprise about 0.005 to 10%, preferably about 0.01 to 5%, more preferably about 0.1 to 5%, by weight of the high molecular weight water soluble polymer(s). For example, aqueous shaving compositions may include about 0.05 to 3%, preferably about 0.1 to 2%, more preferably about 0.1 to 1%, by weight polyethylene oxide and about 0.1 to 5%, preferably about 0.1 to 2%, more preferably about 0.2 to 1.5%, by weight polysaccharide gum.

The shaving compositions of the present invention may be formulated as simple aqueous solutions that can be applied to the face prior to shaving. Advantageously, other cosmetic ingredients may be added to improve the application aesthetics and/or achieve other shave benefits. For example, the composition may include one or more of the following components: beard wetting agents, skin conditioning agents (e.g. vitamins A, C and E, aloe, allantoin, panthenol, alpha-hydroxy acids, phospholipids, triglycerides, botanical oils, amino acids), cleansing agents, lathering agents, foaming agents, emollients, humectants (e.g., glycerin, sorbitol, propylene glycol), water dispersible surface active agents (e.g., soaps (including interrupted soaps), detergents, non-ionic, anionic and amphoteric surfactants), gelling or thickening agents, propellants or self-foaming agents, fragrances, colorants, antioxidants, preservatives, etc.

Thus, one aspect of the present invention embraces a shaving composition comprising water, a cleansing or conditioning agent for hair or skin, and at least one water soluble polymer, wherein the composition has a stress ratio in excess of 3.0, preferably in excess of 4.0, more preferably in excess of 5.0. As mentioned previously, normal stress and shear stress are measured at (or interpolated to) a shear rate of 800 sec⁻¹. The composition should be measured without propellant, either prior to addition of propellant, which is preferred, or after evaporation or removal of propellant from the finished product

Another aspect of the present invention embraces a shaving composition comprising water, a cleansing or conditioning agent for hair or skin, a polyethylene oxide having a molecular weight of one million or higher, and a natural or synthetic gum (e.g., a polysaccharide gum, particularly one selected from the group consisting of xanthan gum, carrageenan gum, guar gum, locust bean gum and hydroxypropyl guar gum).

Preferably, the composition will be in the form of a shaving lotion, cream, foam or gel. Such a formulation will typically comprise, by weight, about 60% to 95%, preferably about 70% to about 90%, water and about 3% to 25%, preferably about 5% to 20%, of a water dispersible (or soluble) surface active agent. The surface active agent may include one or more water soluble soaps (including interrupted soaps), detergents, anionic surfactants, amphoteric surfactants and/or non-ionic surfactants. Naturally, of course, the shaving composition may contain a variety of well-known cosmetic ingredients which are typically used to enhance the performance attributes and aesthetics thereof.

A preferred shaving composition of the present invention comprises about 60% to 95% water, about 3% to 25% of a water dispersible surface active agent, and about 0.005% to 10%, preferably about 0.01% to 5%, of at least one water soluble polymer, preferably two polymers, wherein the shaving composition has a stress ratio in excess of 3.0, preferably in excess of 4.0, more preferably in excess of 5.0.

The water dispersible surface active agent may comprise a soap, a detergent, an anionic surfactant, a non-ionic surfactant, or a mixture of one or more of these. The soaps include, for example, the sodium, potassium and lower alkanolamine (preferably triethanolamine) salts of C₁₀ to C₂₀, preferably C₁₂ to C₁₈, fatty acids. Typical fatty acids include lauric, oleic, coconut oil, myristic, palmitic and stearic acid and mixtures thereof. The preferred fatty acids are palmitic and stearic. For purposes of the present invention, the water dispersible soaps are also intended to include the interrupted soaps such as the sodium, potassium and lower alkanolamine (preferably triethanolamine) salts of N-fatty acyl sarcosines wherein the fatty acyl moiety has 10 to 20, preferably 12 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps (including the interrupted soaps) may be utilized in preneutralized form (i.e., as the sodium, potassium or alkanolamine salt) or in the free acid form followed by subsequent neutralization with sodium hydroxide, potassium hydroxide and/or alkanolamine (preferably triethanolamine). In any event, the composition must contain sufficient base to neutralize or partially neutralize the soap component and adjust the pH to the desired level.

The water dispersible surface active agent may also optionally include a non-ionic, amphoteric and/or anionic surfactant. Suitable non-ionic surfactants will typically have an HLB of 14 or more and include the polyoxyethylene ethers of fatty alcohols, acids and amides, particularly those having 10 to 20, preferably 12 to 18, carbon atoms in the fatty moiety and about 8 to 60, preferably 10 to 30, ethylene oxide units. These include, for example, PEG-150 Distearate, Oleth-20, Steareth-21, Ceteth-20, and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, lauryl polyglucoside, sucrose laurate, and polyglycerol 8-oleate. Suitable amphoteric surfactants include, for example, the betaines and sultaines such as cocoamidopropyl betaine, coco dimethyl carboxymethyl betaine, coco sultaine and the like. Suitable anionic surfactants include, for example, the sodium, potassium, ammonium and substituted ammonium salts (such as the mono-, di- and triethanolamine salts) of C₈-C₂₂, preferably C₁₂-C₁₈, alkyl sulfates (e.g. sodium lauryl sulfate, ammonium lauryl sulfate), alkyl sulfonates (e.g. ammonium lauryl sulfonate), alkylbenzene sulfonates (e.g. ammonium xylene sulfonate), acyl isethionates (e.g. sodium cocoyl isethionate), acyl lactylates (e.g. sodium cocoyl lactylate) and alkyl ether sulfates (e.g. ammonium laureth sulfate). The surface active agent may typcially include up to about 8% of non-ionic, amphoteric and/or anionic surfactants.

In addition to the surface active agent, the shaving composition may optionally include a variety of other well-known cosmetic ingredients generally known for use in shaving creams, foams and gels to improve the aesthetics and performance characteristics of the composition.

The shaving composition may contain about 1% to 10%, preferably about 1.5% to 7%, of a non-volatile paraffinic hydrocarbon fluid. The terms “non-volatile” and “fluid” mean that these materials are liquid at room temperature and have a boiling point above 200° C. Such hydrocarbon fluids include mineral oils and branched-chain aliphatic liquids. These fluids typically have from about 16 to about 48, preferably about 20 to about 40, carbon atoms and a viscosity of about 5 to about 100 cs., preferably about 10 to about 50 cs., at 40° C. The preferred non-volatile paraffinic hydrocarbon fluid is selected from mineral oil with a viscosity of about 10 to about 50 cs. at 40° C., hydrogenated polyisobutene with a molecular weight of about 320 to about 420, and mixtures thereof.

It may also be desirable to include a water-soluble gelling aid or thickening agent in the shaving composition to improve its consistency and stability, as well as to adjust its viscosity. These may include, for example, hydroxyalkyl cellulose polymers such as hydroxyethyl cellulose and hydroxypropyl cellulose (sold under the trademarks “Natrosol” and “Klucel” respectively), copolymers of acrylic acid and polyallyl sucrose (sold under the trademark “Carbopol”), carboxymethyl cellulose, and cellulose methyl ether (sold under the trademark “Methocel”). The gelling aid or thickening agent is typically included in an amount of about 0.01% to 5%, preferably about 0.1% to 2%, by weight of the composition. Generally, however, the compositions of the present invention do not need any additional gelling or thickening agents because the polymers utilized in accordance with the present invention to provide stress ratios in excess of 3.0 sufficiently thicken the composition.

The shaving composition may also include up to 8%, preferably about 2% to 6%, by weight of a fatty alcohol such as myristyl, lauryl and stearyl alcohol and octyl dodecanol. The term “fatty” is intended to include 10 to 20, preferably 12 to 18, carbon atoms.

Other useful additives which may be utilized in the composition include humectants such as glycerin, sorbitol, and propylene glycol, emollients including fatty esters such as isopropyl myristate, decyl oleate, 2-ethylhexyl palmitate, PEG-7 glyceryl cocoate, and glyceryl linoleate, propoxylated fatty ethers such as PPG-10 cetyl ether and PPG-11 stearyl ether, di- and triglycerides such as lecithin and caprylic/capric triglyceride, vegetable oils, and similar materials, skin freshening and soothing agents such as menthol, aloe, allantoin, lanolin, collagen and hyaluronic acid, fluorosurfactants, silicones (e.g. dimethicone, dimethiconol, dimethicone copolyol, stearyl dimethicone, cetyl dimethicone copolyol, phenyl dimethicone, cyclomethicone, etc.), vitamins (including vitamin precursors and derivatives) such as panthenol, vitamin E, tocopherol acetate, and vitamin A palmitate, colorants, fragrances, antioxidants and preservatives.

If the shaving composition is in the form of a self-foaming shave gel, it will include a self-foaming agent which may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred self-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1. The self-foaming agent will normally be present in an amount comprising about 1% to about 6% of the composition, preferably about 2% to about 5%.

If the shaving composition is in the form an aerosol foam, it will include a propellant of sufficient volatility or pressure to propel the shaving composition from its container and cause it to foam. Typical propellants include compressed air and, more typically, a volatile hydrocarbon or halohydrocarbon or mixture of hydrocarbons (typically with 3 to 6 carbon atoms) having a vapor pressure of 30 to 60 psig at about 20° C. A preferred propellant has the industry designation A-46 and is a mixture of n-butane, isobutane and propane with a vapor pressure of 46 psig at about 20° C. Other suitable propellants include propellant 152A and A-70. When the propellant is a volatile hydrocarbon, it typically comprises about 1% to 5%, preferably about 2% to 4%, of the composition.

One type of shaving composition of the present invention is an aerosol shaving foam which comprises, in percent by weight, about 65% to 90% (preferably about 70% to 90%) water, about 3% to 25% (preferably about 5% to 20%) of a water dispersible surface active agent, a sufficient amount of water soluble polymer(s) to provide the composition with a stress ratio in excess of 3.0, and propellant (preferably about 1% to 5% volatile hydrocarbon). In one embodiment, the polymers will include, for example, about 0.1% to 2% (preferably about 0.1% to 1%) polyethylene oxide (M.Wt.=1-5 million) and about 0.1% to 2% (preferably about 0.2% to 1.5%) natural or synthetic gum (e.g., polysaccharide gum).

Another type of shaving composition of the present invention is a non-aerosol shave gel which comprises, in percent by weight, about 65% to 95% (preferably about 70% to 90%) water, about 3% to 25% (preferably about 5% to 20%) of a water dispersible (or soluble) surface active agent, and a sufficient amount of water soluble polymer(s) to provide the composition with a stress ratio in excess of 3.0. In one embodiment, the polymers will include, for example, about 0.1% to 2% (preferably about 0.1% to 1%) polyethylene oxide (M.Wt.=1-5 million) and about 0.1% to 2% (preferably about 0.2% to 1.5%) natural or synthetic gum (e.g., polysaccharide gum).

A further type of shaving composition of the present invention is a self-foaming shave lotion or gel which comprises, in percent by weight, about 65% to 95%, (preferably about 70% to 90%) water, about 3% to 25% (preferably about 5% to 20%) of a water dispersible (or soluble) surface active agent, a sufficient amount of water soluble polymer(s) to provide the composition with a stress ratio in excess of 3.0, and about 1% to 6% (preferably about 2% to 5%) self-foaming agent (e.g., volatile hydrocarbon). In one embodiment, the polymers will include, for example, about 0.1% to 2% (preferably about 0.1% to 1%) polyethylene oxide (M.Wt.=1-5 million) and about 0.1% to 2% (preferably about 0.2% to 1.5%) natural or synthetic gum (e.g., polysaccharide gum).

The shaving compositions of the present invention may be packaged in any suitable dispenser normally used for dispensing shaving lotions, creams, foams or gels. These include aerosol dispensers in which the propellant is added to the same chamber as the shaving concentrate (e.g., as is typically done with shaving foams), aerosol dispensers with a barrier, such as a collapsible bag or piston, to separate the shaving concentrate from the propellant required for expulsion (e.g., as is typically done with self-foaming gels), collapsible tubes, and pump or squeeze containers. It is preferred to protect the composition from oxidation and heavy metal contamination. This can be achieved, for example, by purging the composition and container with nitrogen to remove oxygen and by utilizing inert containers (e.g., plastic bottles, aluminum cans or polymer coated or lined cans).

The present invention is also directed to an improved shaving method in which a shaving composition of the present invention is applied to an area of skin, then said area is shaved, preferably with a wet razor. In particular, the invention embraces a method of shaving comprising applying to an area of skin an aqueous polymer solution having a stress ratio in excess of 3.0, preferably in excess of 4.0, more preferably in excess of 5.0, when measured at (or interpolated to) a shear rate of 800 sec⁻¹, and shaving said area of skin. The invention further embraces a method of shaving comprising applying to an area of skin a shaving composition and shaving said area of skin, wherein said shaving composition comprises one or more polyethylene oxides of molecular weight of about one million to about five million and one or more natural or synthetic gums (e.g., polysaccharide gums, particularly those selected from the group consisting of xanthan gum, carrageenan gum, guar gum, locust bean gum and hydroxypropyl guar gum). The shaving composition may be an aqueous polymer solution, such as a shave lotion, cream, foam or gel.

The invention may be further described by the following examples in which all parts and percentages are by weight.

### EXAMPLES 1-5

### Non-Aerosol Shave Composition

| | Weight Percent | | | | |
|---|---|---|---|---|---|
| Ingredient | Ex. 1 | Ex. 2 | Ex. 3 | Ex. 4 | Ex. 5 |
| Carageenan ¹ | 2.5 | | | | 1.0 |
| NatrosoL 250 HHR | | | | 1.4 | |
| Carbopol EDT-2020 | | 1.0 | 0.75 | | |
| KOH | | | | 7.68 | |
| Triethanolamine | | 1.5 | 6.58 | | 2.85 |
| Sod. Lauroyl Sarcosinate | | | | 3.75 | |
| Sod. C₁₄₋₁₆ Olefin Sulfonate | 5.0 | 5.0 | 5.0 | | 5.0 |
| Polyquaternium-22 | | 1.0 | 1.0 | | |
| Polyquaternium-39 | 5.0 | | | | |
| PEG-23M ² | 0.11 | 0.11 | 0.22 | 0.11 | |
| PEG-90M ³ | 0.008 | 0.004 | 0.008 | 0.004 | 0.15 |
| Perservative | 0.25 | 0.25 | 0.27 | | 0.27 |
| Myristic Acid | | | 6.67 | 10.0 | 4.40 |
| Palmitic Acid | | | | 5.0 | |
| Laureth-23 (Brij 35SP) | | | 1.00 | | 1.0 |
| Oleth-20-VS (Brij 98 VS) | | | | 3.0 | |
| Cocamidopropyl Betaine and | | | 3.00 | | 3.0 |
| Cocamide MEA | | | | | |
| PEG-150-DS | | | 1.0 | 1.5 | 1.0 |
| Water | q.s. | q.s | q.s | q.s. | q.s. |

¹ Genuvisco TPH-1 (Aqualon)
² WSRN-12K (Union Carbide; M.Wt. ≅ 1,000,000)
³ WSRN-301 (Union Carbide; M.Wt. ≅ 4,000,000)

The above-described compositions are made in the following manner: For Ex. 1 and 2, preblend the polymers, disperse into water, then mix until complete dissolution (about 1 hr.). Add remaining ingredients and mix for 20 minutes, then fill into squeeze bottles. For Ex. 3 to 5, preblend the polymers, disperse into water, mix until complete dissolution (about 1 hr.) and heat to 75° C. At 60° C., add myristic/palmitic acid, laureth-23 and PEG-150-DS and mix until melted. Add triethanolamine or KOH and mix for 20 minutes, then cool to 30° C. Add remaining ingredients, mix for 10 minutes and fill into squeeze bottles.

### EXAMPLES 6-9

### Aerosol Shave Foam

| | Weight Percent | | | |
|---|---|---|---|---|
| Ingredient | Ex. 6 | Ex. 7 | Ex. 8 | Ex. 9 |
| Palmitic acid | 6.31 | 6.31 | 6.31 | 6.29 |
| Triethanolamine | 3.40 | 3.40 | 3.40 | 3.39 |
| Laureth-23 | .97 | 1.07 | 1.46 | 4.35 |
| Sodium lauryl sulfate (30%) | 2.62 | 2.62 | 2.62 | 3.87 |
| PEG-150 distearate | 0.24 | 0.24 | 0.24 | 0.29 |
| Nonoxynol-9 | 0.49 | 0.49 | 0.97 | 0.97 |
| Stearyl alcohol | | | 0.58 | 1.55 |
| Fragrance | 0.38 | 0.38 | 0.38 | 0.38 |
| BHT | 0.02 | 0.02 | 0.02 | 0.02 |
| Sodium benzoate | 0.24 | 0.24 | 0.24 | 0.24 |
| Quaternium-15 | 0.03 | 0.03 | 0.03 | 0.03 |
| Tocopheryl acetate | 0.02 | 0.02 | 0.19 | 0.19 |
| Propellant A-46 | 2.88 | 2.88 | 2.88 | 3.24 |
| Carrageenan ¹ | 0.68 | 0.68 | 0.68 | 0.10 |
| PEG-90M ² | 0.15 | 0.03 | 0.02 | |
| PEG-45M ³ | | 0.06 | 0.19 | 0.19 |
| PEG-23M ⁴ | | 0.10 | | |
| Water | q.s. | q.s. | q.s. | q.s. |

¹ Genuvisco TPH-1 (Hercules - Aqualon Corp.)
² WSR-301 (Union Carbide; M.Wt. ≅ 4,000,000)
³ WSR N-60K (Union Carbide; M.Wt. ≅ 2,000,000)
⁴ WSR N-12K (Union Carbide; M.Wt. ≅ 1,000,000)

The above-described compositions are made in the following manner: Preblend the polymers, disperse into water, mix until complete dissolution (about 1 hr.) and heat to 75° C. At 60° C., add palmitic acid, laureth-23, stearyl alcohol, and PEG-150-DS and mix until melted. Add triethanolamine and mix for 20 minutes. Add BHT, mix for 10 minutes. Add sodium lauryl sulfate and mix for 10 minutes. Cool to 30° C. Add sodium benzoate and Quaternium-15 and mix for 10 minutes. Preblend fragrance, vitamin E and nonoxynol-9 and add to batch. Add remaining water and mix for 10 minutes. Add concentrate to aerosol can, then fill with propellant.

### EXAMPLES 10-14

### Self-Foaming Shave Gel

| | Weight Percent | | | | |
|---|---|---|---|---|---|
| Ingredient | Ex. 10 | Ex. 11 | Ex. 12 | Ex. 13 | Ex. 14 |
| Palmitic acid | 8.16 | 8.16 | 8.16 | 8.16 | 7.76 |
| Triethanolamine | 5.00 | 5.00 | 5.00 | 5.00 | 4.75 |
| Oleth-20 | 5.28 | 5.28 | 5.28 | 5.28 | 2.64 |
| Cocamide MEA | | | | | 1.44 |
| Hydrog. Polyisobutene¹ | 1.92 | 1.92 | 1.92 | 1.92 | 1.92 |
| Glycerin | 1.92 | 1.92 | 1.92 | 1.92 | 1.92 |
| Lauramide DEA | 1.44 | 1.44 | 1.44 | 1.44 | |
| Isopentane/isobutane (3:1) | 3.85 | 3.85 | 3.85 | 3.85 | 3.85 |
| Fluorosurfactant² | 0.01 | 0.01 | 0.01 | 0.01 | 0.01 |
| Fragrance/dye | 1.05 | 1.05 | 1.05 | 1.05 | 0.97 |
| PEG-150 distearate | 0.24 | 0.24 | 0.24 | 0.24 | 0.24 |
| Xanthan gum³ | | | 0.75 | | |
| Carrageenan⁴ | 0.48 | 0.29 | | | 0.48 |
| Guar gum⁵ | | | | 0.72 | |
| PEG-45M⁶ | 0.19 | | 0.24 | 0.24 | 0.19 |
| PEG-90M⁷ | | 0.14 | | | |
| Tocopheryl acetate | | | | | 0.14 |
| PEG-20 methyl glucose sesquistearate⁸ | | | | | 0.24 |
| Water | q.s. | q.s. | q.s. | q.s. | q.s. |

¹Panalane L14-E (Amoco)
²Zonyl FSK (Dupont), added as 1% aqueous solution
³Keltrol CG-T (Kelco)
⁴Genuvisco TPH-1 (Hercules - Aqualon Corp.)
⁵Aqualon Supercol Guar (Aqualon)
⁶WSR N-60K (Union Carbide; M.Wt. ≅ 2,000,000)
⁷WSR-301 (Union Carbide; M.Wt. ≅ 4,000,000)
⁸Glucamate SSE-20 (Amerchol)

The above-described compositions are made in the following manner: The gum and polyethylene oxide are dispersed in glycerin, then added to water and mixed well (about 30 min.). The mixture is heated, then at 60° C. the palmitic acid, Panalane and Glucamate SSE-20 are added. After the palmitic acid is melted and the temperature is 80-85° C., triethanolamine is added and mixed for about 40 minutes. The Oleth-20, Cocamide MEA and PEG-150 distearate are separately mixed and melted at about 73° C., then added to the soap phase and mixed for about 30 minutes. While cooling this mixture to 30° C., the remaining ingredients are added at about 45° C. and mixed well. The concentrate (at about 20° C.) is mixed with the volatile self-foaming agent (at about 5° C.) in a pressurized vessel until slightly gelled, then filled into bottom-gassed cans.

### EXAMPLES 15-18

### Self-Foaming Shave Lotion

| | Weight Percent | | | |
|---|---|---|---|---|
| Ingredient | Ex. 15 | Ex. 16 | Ex. 17 | Ex. 18 |
| Palmitic acid | 8.54 | | | 6.57 |
| Stearic acid | | | | 0.98 |
| Triethanolamine (99%) | 5.23 | 0.72 | 3.95 | 4.18 |
| Glycerin | 2.11 | | 2.11 | |
| Sod. Olefin C₁₄₋₁₆ Sulfonate ¹ | | 2.40 | | |
| Stearoyl/Myristoyl Sarcosine ² | | | 10.78 | |
| Isopentane/isobutane (4:1) | 3.85 | 3.85 | 3.85 | 2.00 |
| Carrageenan ³ | 0.53 | | 0.53 | 0.98 |
| Carbopol EDT 2020 | | 0.48 | | |
| PEG-45M ⁴ | 0.21 | | 0.21 | 0.11 |
| PEG-23M ⁵ | | 0.16 | | |
| Polyquaternium-22 ⁶ | | 0.48 | | |
| Laurylamidopropyl Betaine ⁷ | | | | 6.80 |
| Polyquaternium-39 ⁸ | | | | 4.90 |
| Oleth-20 | | | | 1.47 |
| Lauramide DEA | | | | 0.98 |
| Water | 79.53 | 91.91 | 78.57 | 71.03 |

¹ BioTerge AS 40 (Stephan Chemical Co.)
² Hamposyl SM Sarcosine (Hampshire Chemical Inc.)
³ Genuvisco TPH-1 (Hercules - Aqualon Corp.)
⁴ WSR N-60K (Union Carbide; M.Wt. ≅ 2,000,000)
⁵ WSR N-12K (Union Carbide; M.Wt. ≅ 1,000,000)
⁶ Merquat 280 (Calgon Corp.)
⁷ Amphosal LB (36%, Stephan Chemical Co.)
⁸ Merquat 3330 (Calgon Corp.)

The above-described compositions are made in the following manner: The water soluble components (i.e., polymers (gum, polyethylene oxide, Carbopol), glycerin, sorbitol) are added to water and mixed until the polymers are completely dissolved (about 30 min.). The mixture is then heated and the palmitic acid (or pre-melted sarcosine or sulfonate) is added at about 60° C. and well mixed while the heating continues. At 80-85° C. the triethanolamine is added and mixed for about 40 minutes. While cooling the soap phase, the phospholipids, beeswax and PEG-150 distearate (and vitamin E if used) are separately mixed at about 65° C. until melted, then added to the soap phase and mixed for about 30 minutes. While cooling this mixture to 30° C., the remaining ingredients are added at about 45° C. and mixed well. The concentrate (at about 20° C.) is then mixed with the volatile self-foaming agent (at about 5° C.) in a pressurized vessel, then filled into bottom-gassed cans.

While particular embodiments of the invention have been shown and described for illustrative purposes, it will be apparent to those skilled in the art that various changes and modifications may be made without departing from the spirit and scope of the invention, which is defined by the claims which follow.

What is claimed is:

1. A method of shaving comprising applying to an area of skin a shaving composition and shaving said area of skin, wherein the shaving composition comprises an aqueous polymer solution having a stress ratio in excess of 3.0 at a shear rate of 800 sec⁻¹.

2. The method of claim 1 wherein the shaving composition comprises an aqueous polymer solution having a stress ratio in excess of 4.0.

3. The method of claim 1 wherein the shaving composition comprises an aqueous polymer solution having a stress ratio in excess of 5.0.

4. A method of shaving comprising applying to an area of skin a shaving composition and shaving said area of skin, wherein the shaving composition comprises an aqueous solution comprising at least one polyethylene oxide of molecular weight of about one million to about five million and at least one polysaccharide gum.

5. The method of claim 4 wherein the polysaccharide gum is selected from the group consisting of xanthan gum, carrageenan gum, guar gum, locust bean gum and hydroxypropyl guar gum.

6. The method of claim 5 wherein the shaving composition is in the form of a shaving lotion, cream, foam or gel.

7. The method of claim 1, 4, 5 or 6 wherein the shaving composition comprises, by weight, about 0.05% to 3% polyethylene oxide and about 0.1% to 3% polysaccharide gum.

8. The method of claim 1, 4, 5 or 6 wherein the shaving composition comprises, by weight, about 0.1% to 2% polyethylene oxide and about 0.1% to 2% polysaccharide gum.

9. A method of increasing the lubricity of an aqueous shaving composition which comprises adding at least one water soluble polymer to the shaving composition, wherein the at least one water soluble polymer provides the composition with a stress ratio in excess of 3.0 at a shear rate of 800 sec⁻¹.

10. The method of claim 9 wherein the at least one water soluble polymer provides the composition with a stress ratio in excess of 4.0.

11. The method of claim 9 wherein the at least one water soluble polymer comprises two polymers.

12. The method of claim 11 wherein the two polymers comprise at least one polyethylene oxide of molecular weight of about one million to about five million and at least one polysaccharide gum.

13. The method of claim 12 wherein the polysaccharide gum is selected from the group consisting of xanthan gum, carrageenan gum, guar gum, locust bean gum and hydroxypropyl guar gum.

14. The method of claim 13 wherein the two polymers are added in an amount such that the shaving composition will comprise, by weight, about 0.05% to 3% polyethylene oxide and about 0.1% to 5% polysaccharide gum.

15. The method of claim 13 wherein the two polymers are added in an amount such that the shaving composition will comprise, by weight, about 0.1% to 1% polyethylene oxide and about 0.2% to 1.5% polysaccharide gum.

16. A shaving composition comprising water, a cleansing or conditioning agent for hair or skin, and at least one water soluble polymer, wherein the composition has a stress ratio in excess of 3.0.

17. The shaving composition of claim 16 having a stress ratio in excess of 4.0.

18. The shaving composition of claim 16 having a stress ratio in excess of 5.0.

19. The shaving composition of claim 16 wherein the at least one water soluble polymer comprises two polymers.

20. The shaving composition of claim 16 comprising, by weight, about 60% to 95% water, about 3% to 25% of a water dispersible surface active agent, and about 0.01 to 10% of the at least one water soluble polymer.

21. The shaving composition of claim 16 comprising, by weight, about 70% to 90% water, about 5% to 20% of a water dispersible surface active agent, and about 0.1% to 5% of the at least one water soluble polymer.

22. The shaving composition of claim 16, 17, 18, 19, 20 or 21 wherein the at least one polymer comprises at least one polyethylene oxide of molecular weight of about one million to about five million and at least one polysaccharide gum.

23. A shaving composition comprising water, a cleansing or conditioning agent for hair or skin, and at least one polyethylene oxide of molecular weight of about one million to about five million and at least one polysaccharide gum.

24. The shaving composition of claim 23 wherein the polysaccharide gum is selected from the group consisting of xanthan gum, carrageenan gum, guar gum, locust bean gum and hydroxypropyl guar gum.

25. The shaving composition of claim 23 or 24 comprising, by weight, about 60% to 95% water, about 3% to 25% of a water dispersible surface active agent, about 0.05% to 3% polyethylene oxide and about 0.1% to 5% polysaccharide gum.

26. The shaving composition of claim 24 comprising, by weight, about 70% to 90% water, about 5% to 20% of a water dispersible surface active agent, about 0.1% to 1% polyethylene oxide and about 0.1% to 2% polysaccharide gum.

27. The shaving composition of claim 16, 20, 23 or 26 in the form of a lotion, cream, foam or gel.

28. The shaving composition of claim 16, 20, 23 or 26 additionally comprising a volatile self-foaming agent.
